(19) United States
(12) Patent Application Publication
(10) Pub. No.: US 2026/0053670 A1
(43) Pub. Date: Feb. 26, 2026

(54) USE OF PENETRATING SCHLEMM’S CANALOTOMY IN CHILDHOOD GLAUCOMA

(71) Applicant: Qingdao Eye Hospital of Shandong First Medical University (Shandong Eye Institute), Qingdao (CN)

(72) Inventors: Xiaojing PAN, Qingdao (CN); Longfang Zhou, Qingdao (CN); Die Hu, Qingdao (CN); Qingshu Ge, Qingdao (CN); Jingyi Wu, Qingdao (CN); Jie Lan, Qingdao (CN)

(21) Appl. No.: 18/813,166

(22) Filed: Aug. 23, 2024

(51) Int. Cl. A61F 9/007; A61B 17/3205

(52) U.S. Cl. CPC A61F 9/00781; A61B 17/32053

(57) ABSTRACT

The present disclosure belongs to the technical field of ophthalmology department and particularly relates to use of a penetrating Schlemm's canalotomy in childhood glaucoma. The present disclosure integrates 360° Schlemm's canalotomy with ab externo trabeculectomy to develop a penetrating Schlemm's canalotomy, thereby improving the postoperative success rate and reducing the adverse effects of transient elevation of intraocular pressure on visual function. This approach provides a better and more stable pressure-reduction for child patients.

### TECHNICAL FIELD

The present disclosure belongs to the technical field of ophthalmology department and particularly relates to the application of a penetrating Schlemm's canalotomy for the treatment of childhood glaucoma.

### BACKGROUND

Childhood glaucoma comprises a heterogeneous group of diseases characterized by elevated intraocular pressure damages to ocular structures. Children with this condition often suffer from poor visual prognosis due to ametropia and vision development disorder, posing serious threats to their vision and making diagnosis and treatment relatively difficult. Childhood glaucoma includes primary congenital glaucoma (PCG), secondary glaucoma occurring between ages 0-18, and mixed glaucoma occurring between ages 0-18. In the United States, there are 2.29 cases of childhood glaucoma per 100,000 residents under age 20, with secondary glaucoma (1.46/100,000) being more prevalent than PCG (0.38/100,000). In the United Kingdom, there are 5.41 cases of PCG per 100,000 live births. The causes of secondary childhood glaucoma include lens factors (31%), phakomatosis (23%), uveitis (19%), and anterior segment dysgenesis (10%). Childhood glaucoma represents approximately 5% of blinding eye diseases in children, 1.2% in the UK, and up to 7% in south India. An epidemiological investigation in schools for the blind in the Beijing area of China shows that the primary congenital glaucoma accounts for nearly 15% of blindness cases. The complexity of childhood glaucoma and the physiological characteristics of children greatly increase the treatment difficulty. Current treatments face great challenges: medications often have significant side effects and limited efficacy, and surgical outcomes are inconsistent. Particularly for children with advanced glaucoma, more effective surgical interventions are necessary to lower intraocular pressure and preserve residual visual function.

The treatment of childhood glaucoma faces many challenges, including disease heterogeneity, difficulties in diagnosis and follow-up, and a long course of disease may require multiple operations throughout the infant's life. Consequently, many children struggle to develop and maintain good vision and visual function. In the treatment of childhood glaucoma, medication serves primarily as an adjunct, used mainly for temporary intraocular pressure control during the perioperative period of PCG, for children who cannot undergo surgery due to systemic diseases, and as the first-line treatment of secondary childhood glaucoma. Surgery is the main treatment for childhood glaucoma. Common surgical options include goniotomy, trabeculotomy, trabeculectomy, glaucoma drainage device implantation, cyclodestructive procedures, external filtration surgery, and deep sclerectomy. In recent years, 360° Schlemm's canalotomy, performed with the assistance of an illuminating microcatheter, has shown improved success rates. However, transient postoperative intraocular pressure elevation may worsen visual function in patients with advanced childhood glaucoma. Ab externo trabeculectomy is currently a primary surgical approach for childhood glaucoma, but due to the thick Tenon capsule tissue in children and their robust proliferative and healing responses, the long-term success rate of a straightforward trabeculectomy is diminished. Therefore, effective management of childhood glaucoma and accurate assessment and improvement of visual prognosis remain long-standing challenges in the field of global glaucoma research.

### SUMMARY

The present disclosure integrates 360° Schlemm's canalotomy with ab externo trabeculectomy to develop a penetrating Schlemm's canalotomy, thereby improving the postoperative success rate and reducing the adverse effects of transient elevation of intraocular pressure on visual function. This approach provides a better and more stable intraocular pressure reduction for child patients.

In a first aspect, the present disclosure provides a surgical method for penetrating Schlemm's canalotomy, comprising the following steps:
S1, performing anesthesia: performing anesthesia by retrobulbar anaesthesia;
S2, fixing an eyeball: pulling a superior rectus by using a 5-0 silk suture, and for a patient with a larger diameter of cornea, suspending a corneal limbus by using a 6-0 absorbable suture;
S3, creating a conjunctival flap: forming a conjunctival flap with a fornix as a base, cutting a full layer of bulbar conjunctiva and a subconjunctival tissue at the corneal limbus along the corneal limbus, performing a blunt dissection by closely attaching the surface of a sclera under a Tenon capsule to expose a surgical field, and performing a blunt dissection from an incision to a conjunctival fornix;
S4, performing hemostasis in a surgical area: performing hemostasis on the sclera surface of the intended scleral flap area by using an underwater electrocoagulation method to avoid excessive electrocoagulation;
S5, creating a superficial scleral flap: creating a superficial scleral flap based at the corneal limbus, measuring 4 mm×3 mm and approximately half the thickness of the sclera; dissecting forward to the transparent corneal limbus, performing hemostasis in the scleral flap area again and placing mitomycin under the conjunctiva and the scleral flap;
S6, puncturing an anterior chamber: making an auxiliary incision in the transparent cornea within the corneal limbus in the upper nasal or upper temporal area by using a puncture knife to keep intraocular pressure at slightly lower than normal level; and injecting a miotic and a small amount of a viscoelastic agent into the anterior chamber to reduce the risk and degree of outward bulging of the inner wall of Schlemm's canal;
S7, making a deep scleral flap: making a deep scleral flap in the central area of a scleral bed beneath the superficial scleral flap and gradually dissecting forward; and when ring-like fibers appear in the scleral bed, continuing the dissection until the outer wall of Schlemm's canal is incised, exposing the gray-brown inner wall of Schlemm's canal;
S8, penetrating the Schlemm's canal: connecting a microcatheter with an illumination indicator to a viscoelastic injector and rotating the injector until the viscoelastic agent emerge from the tip of the microcatheter; dimming the microscope light, gently clamping the microcatheter by a pair of microscopic forceps, inserting the microcatheter into an open end of the Schlemm's canal, slowly advancing the microcatheter along the corneal limbus, and monitoring the position of indicator lamp at the tip of the microcatheter to ensure it remains within the lumen of Schlemm's canal until the microcatheter exits from the opposite end;
S9, Performing a 360° incision of the Schlemm's canal involves pulling on both ends of an optical fiber to make a 360° cut along the inner wall of the Schlemm's canal. During this procedure, bleeding from the anterior chamber is typically observed.
S10, suturing the deep scleral flap: suturing the deep scleral flap with a 10-0 nylon suture to prevent excessive filtration during and post-surgery, which leads to complications of low intraocular pressure and a shallow anterior chamber;
S11, excising a trabecular tissue: puncturing into the anterior chamber from the anterior edge of the Schlemm's canal under the superficial scleral flap by using a puncture knife and then completely excising corneal and scleral tissues parallel to the corneal limbus;
S12, excising a peripheral iris: making a wide-based peripheral iris excision parallel to the corneal limbus, ensuring the excision area is larger than the trabecular incision;
S13, suturing the scleral flap and the conjunctival flap: the anterior edge of the scleral flap is tightly secured with a 10-0 nylon suture, injecting a balanced salt solution or sterile normal saline into the anterior chamber from the corneal auxiliary puncture site to reform the anterior chamber and checking for fluid leakage at the scleral flap by using a sponge stick or a cotton swab; Finally, the conjunctival incision is sutured using a 10-0 nylon suture in a horizontal mattress fashion;
S14, completing the surgery: removing the superior rectus traction suture or the corneoscleral limbus suspension suture, removing the eyelid speculum, applying antibiotic and glucocorticoid ointment in the conjunctival sac, covering the operated eye with a dressing, and completing the procedure.

Further, in step S1, general anesthesia is administered for a patient with claustrophobia or excessive anxiety.

Further, in step S3, the full layer of the bulbar conjunctiva and the subconjunctival tissue are cut using conjunctival scissors along the limbal, 1 mm outside the corneal limbus. The length is between 6-9 mm, preferably 8 mm. During blunt separation on the scleral surface, the surgical field of 5 mm×4 mm is exposed.

Further, in step S5, the superficial scleral flap is 4 mm×3 mm in size and approximately half the thickness of the sclera.

Further, in step S5, dissecting forward to approximately 0.5-2 mm within the transparent corneal limbus, preferably 1 mm; and mitomycin is placed under the conjunctiva and the scleral flap for 1-4 minutes, preferably 2 minutes.

Further, in step S6, the 15° puncture knife is used as an auxiliary knife to make an auxiliary incision in the transparent cornea 0.5 mm inside the corneal limbus.

Further, in step S7, the deep scleral flap with the size of 1.5 mm×2 mm is made in the central area of the scleral bed below the superficial scleral flap and the depth is such that only a few fibers remain in the scleral bed and the gray-blue choroid membrane can be seen.

Further, in step S8, when the Schlemm's canal is penetrated, if resistance is encountered, the microcatheter can be slightly retracted and a small amount of the viscoelastic agent can be injected to expand the Schlemm's canal and then the microcatheter is advanced continuously; if the microcatheter gets stuck, the microcatheter can be retracted into the Schlemm's canal and pass through again, and if the microcatheter is still stuck, the microcatheter can be withdrawn and reinserted from the opposite end of Schlemm's canal; and if full penetration still cannot be performed, a 360° Schlemm's canalotomy of an internal approach can be considered.

Further, in step S11, the 15° puncture knife punctures in the anterior chamber from the anterior edge of the Schlemm's canal under the superficial scleral flap, and then 1.0 mm×0.5 mm of the corneal and scleral tissues are completely excised in parallel to the corneal limbus.

Further, in step S13, the anterior edge of the scleral flap is tightly sutured with 2 stitches of 10-0 nylon suture. 2 or more adjustable sutures are used for suturing, the balanced salt solution or sterile normal saline are injected into the anterior chamber from the auxiliary puncture site of the cornea to reform the anterior chamber and the fluid leakage is checked at the scleral flap by using a sponge stick or cotton swab; and the conjunctival incision is sutured by using the 10-0 nylon suture in a horizontal mattress manner.

Further, in step S14, a tobramycin dexamethasone eye drop and a tobramycin dexamethasone ointment are routinely used to the operated eye for 4 weeks postoperatively.

### BRIEF DESCRIPTION OF THE DRAWINGS

FIG. 1 illustrates the creation of a conjunctival flap using the fornix as its base;

FIG. 2 depicts the hemostasis of a scleral flap area, achieved through the application of underwater electrocoagulation;

FIG. 3 shows that a superficial scleral flap of approximately 4 mm×3 mm is made by using a 15° knife;

FIG. 4 shows that the superficial scleral flap is made based at the corneal limbus, with a thickness of about half the scleral thickness;

FIG. 5 shows that mitomycin is placed under a conjunctiva and the scleral flap;

FIG. 6 shows that a corneal side incision is made with a 15° knife;

FIG. 7 shows that miotic drugs and viscoelastic agents are injected into the anterior chamber through the side incision;

FIG. 8 shows that a deep scleral flap of about 1.5 mm×2 mm is made under the superficial scleral flap with a 15° knife;

FIG. 9 shows that when the deep scleral flap is dissected forwards, the outer wall of the Schlemm's canal is cut to expose the inner wall of the gray-brown Schlemm's canal

FIG. 10 shows that a microcatheter is clamped by a pair of microscopic forceps and inserting the microcatheter from an open end of the Schlemm's canal;

FIG. 11 shows that the microcatheter penetrates all round along the Schlemm's canal until the microcatheter exits from the opposite end;

FIG. 12 shows that two ends of an optical fiber are pulled and the inner wall of the Schlemm's canal is 360° cut;

FIG. 13 shows that the deep scleral flap is sutured with 10-0 nylon suture and corneal and scleral tissues are completely excised in the corneal limbus;

FIG. 14 shows the excision of a peripheral iris with a wide base;

FIG. 15 shows that the anterior edge of the scleral flap is tightly sutured with 10-0 nylon suture and an adjustable suture is sutured; and

FIG. 16 shows that a conjunctival incision is sutured with 10-0 nylon suture in a horizontal mattress manner.

### DETAILED DESCRIPTION

The following embodiments and technical methods are only used to illustrate the technical solution of the present disclosure and do not limit the scope of the present disclosure. For those skilled in the art, variations or modifications in other different forms can be made on the basis of the above description and idea, and all the embodiments are not necessarily and cannot be listed. Any modification, equivalent replacement or improvement made within the spirit and principle of the present disclosure should be included in the protection scope of the claims of the present disclosure.

The indication of the operation mode is patients with advanced childhood glaucoma. The operation mode has a better long-term effect after being applied to the patients with a visual field MD value <−12 dB and a low risk of early-stage transient ocular intraocular hypertension after the surgery. The specific embodiments show the technical solution of specific operation steps.

### Example 1 Steps of Penetrating Schlemm's Canalotomy

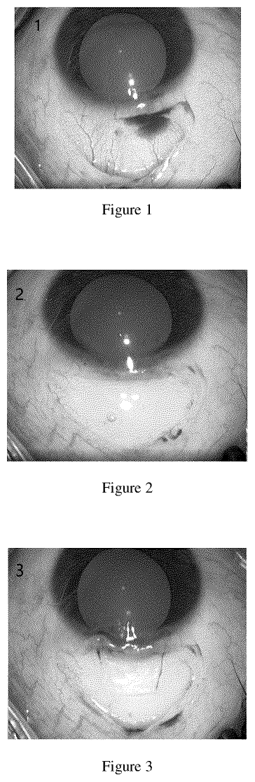

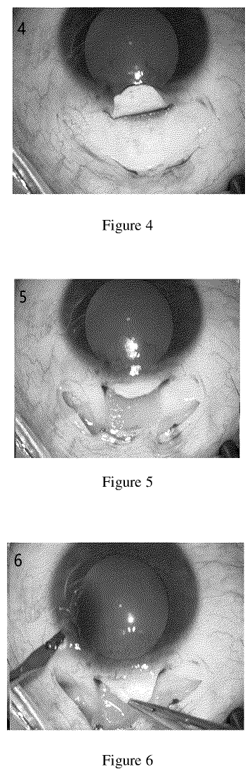

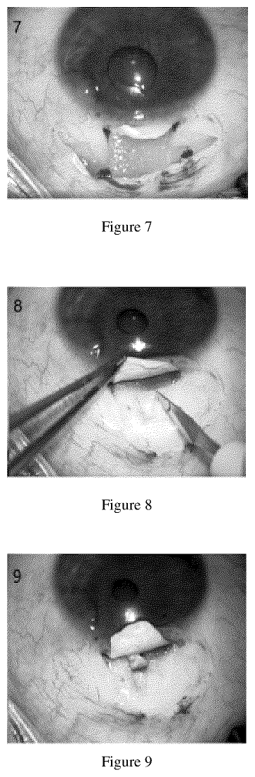

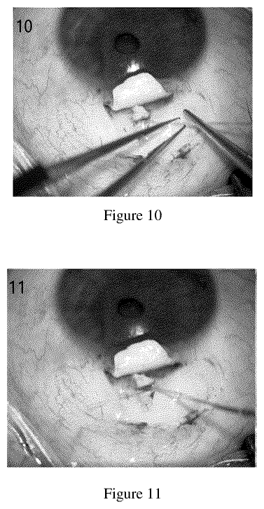

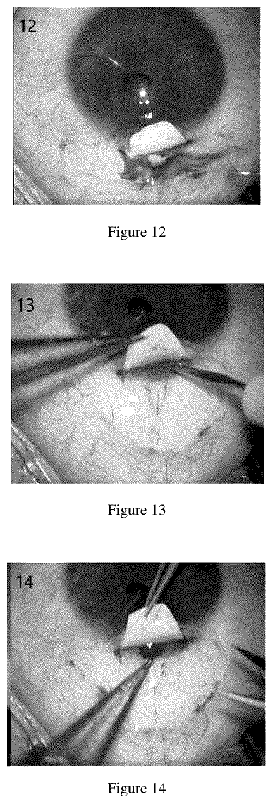

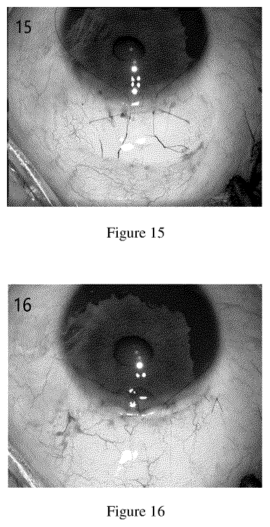

Step 1, anesthesia was performed: anesthesia was performed by retrobulbar anaesthesia and general anesthesia was performed for a child or a patient with claustrophobia or excessive stress.
Step 2, an eyeball was fixed: a superior rectus was pulled by using a 5-0 silk suture, and for a patient with a larger diameter of cornea, a corneal limbus was suspended by using a 6-0 absorbable suture.
Step 3, A conjunctival flap was created, typically using the fornix as a foundation. A full layer of a bulbar conjunctiva and a subconjunctival tissue were cut at a position 1 mm outside the corneal limbus along the radian of the corneal limbus, and the length was 8 mm (FIG. 1). A blunt dissection was performed by closely attaching the surface of a sclera under a Tenon capsule to expose a surgical field with a region about 5 mm×4 mm and blunt dissection was performed from an incision to a conjunctival fornix.
Step 4, hemostasis was performed in a surgical area: hemostasis on the sclera surface of the intended scleral flap area by using an underwater electrocoagulation method to avoid excessive electrocoagulation (FIG. 2).
Step 5, a superficial scleral flap was made: a superficial scleral flap was made by using the corneal limbus as a base with the size of 4 mm×3 mm and the thickness about ½ of the sclera. dissecting forward was performed to 1 mm in the transparent corneal limbus (FIGS. 3 and 4), a scleral flap region was subjected to hemostasis again, and mitomycin was placed under a conjunctiva and the scleral flap for 2 minutes (FIG. 5).
Step 6, an anterior chamber was punctured: an auxiliary incision was made in the transparent cornea position 0.5 mm in the corneal limbus in the upper nasal or upper temporal area by using a 15° puncture knife to keep an intraocular pressure at slightly lower than a normal level and a miotic and a small amount of a viscoelastic agent were injected into the anterior chamber to reduce the risk and degree of outward bulging of the inner wall of a Schlemm's canal (FIGS. 6 and 7).
Step 7, a deep scleral flap was made: a deep scleral flap with the size of 1.5 mm×2 mm was made in the central area of the scleral bed below the superficial scleral flap, the depth was as the criterion when only a few fibers remained in the scleral bed and a gray-blue choroid membrane can be seen, and dissecting forward was gradually performed. Continuous dissecting forward was performed when the scleral bed gradually presented ring-like fibers, namely, the outer wall of the Schlemm's cannel was found to be cut to expose the inner wall of the grayish brown Schlemm's canal (FIGS. 8 and 9).
Step 8, the Schlemm's canal was penetrated: a microcatheter with an illumination indicator to a viscoelastic injector. The injector was rotated until the viscoelastic agent emerged from the tip of the microcatheter. The light of a microscope was dimmed, the microcatheter was gently clamped by a pair of microscopic forceps, the microcatheter was inserted into an open end of the Schlemm's canal and slowly advancing along the corneal limbus, the position of an indicator lamp at the tip of the microcatheter was observed, and whether the microcatheter ran in the lumen of Schlemm's canal or not was determined until the microcatheter exits from the opposite end of the Schlemm's canal. If a resistance was met, the microcatheter can be retracted a little, a small amount of the viscoelastic agent was injected into the Schlemm's canal lumen for expansion and then the microcatheter was pushed forward continuously. If the microcatheter was stuck, the microcatheter can be retracted into the Schlemm's canal and passed through again, and if the microcatheter was still stuck, the microcatheter can be withdrawn and guided to pass from the opposite end of the Schlemm's canal. If full penetration still cannot be performed, a 360° Schlemm's canalotomy of an internal approach can be considered (FIGS. 10 and 11).
Step 9, the Schlemm's canal was incised in a 360° fashion: both ends of an optical fiber was pulled and the inner wall of the Schlemm's canal was 360° cut, wherein anterior chamber bleeding can be generally seen (FIG. 12).
Step 10, the deep scleral flap was sutured: the deep scleral flap was sutured by using a 10-0 nylon suture to prevent the generation of complications of low intraocular pressure and shallow anterior chamber caused by excessive filtration during and after an operation (FIG. 13)
Step 11, a trabecular tissue was excised: the anterior chamber was punctured into from the anterior edge of the Schlemm's canal under the superficial scleral flap by using the 15° puncture knife and then corneal and scleral tissues of 1.0 mm×0.5 mm were completely excised in parallel to the corneal limbus (FIG. 13).
S12, a peripheral iris was excised: a wide-based peripheral iris excision was made parallel to the corneal limbus, wherein an excision area was greater than a trabecular incision range (FIG. 14).
Step 13, the scleral flap was tightly sutured: the anterior end of the scleral flap was tightly sutured by using a 10-0 nylon suture with 2 stitches and 2 or more adjustable sutures were placed. A balanced salt solution or sterile normal saline was injected into the anterior chamber from an auxiliary puncture site a cornea to reform the anterior chamber and the fluid leakage condition at the scleral flap was checked by using a sponge stick or a cotton swab using the stable anterior chamber and no obvious leakage existing at the scleral flap part as the criterion. A 10-0 nylon suture sutured a conjunctival incision in a horizontal mattress manner (FIGS. 15 and 16).
Step 14, the surgery was finished: the superior rectus pulling suture or the corneal limbus suspension suture was removed, an eye speculum was removed, an eye ointment containing an antibiotic and a glucocorticoid was coated in a conjunctival sac, an operated eye was covered with a dressing, and the operation was finished. A tobramycin dexamethasone eye drop and a tobramycin dexamethasone eye ointment were routinely used for 4 weeks after the eye operation specifically according to the state of patients.
   Example 2 Comparison of Penetrating Schlemm's Canalotomy with Common Surgical Methods

### 1. Basic Test Information

### 1.1. Study Objects

40 eyes of 40 cases of children at 0-18 years old diagnosed as childhood glaucoma in Qingdao Eye Hospital between January 2020 and January 2023 were included. Among the children, there were 26 cases of boys and 14 cases of girls. The subjects signed an informed consent.

### 1.2. Inclusion Criteria

(1) Intraocular pressure ≥21 mmHg, with abnormal chamber angle development without other ocular dysgenesis, and optic nerve C/D>0.3 upon fundoscopic examination; (2) Initial diagnosis age of 0-18 years old; (3) Inadequate control of intraocular pressure despite administration of the maximum tolerated doses of intraocular pressure-lowering medications;
(4) No history of other ocular surgeries except anti-glaucoma surgery; and (5) exclusion of patients with secondary glaucoma due to systemic syndromes or other etiologies.

### 1.3. Test Grouping

The patients were randomly divided into three groups, wherein 11 cases of the patients underwent internal drainage surgery, i.e. 360° trabeculotomy with glaucoma gonioscope and microcatheter assistance, 13 cases of the patients underwent external drainage operation, i.e. ab externo trabeculectomy combined with Schlemm's canal outer wall incision, and 16 cases of the patients underwent Schlemm's canalotomy, i.e. the surgical method of the present disclosure.

2. Comparison of Current Common Childhood Glaucoma Surgeries with the Present Disclosure

### 2.1. Internal Drainage Surgery:

In the 360° trabeculotomy with glaucoma gonioscope and microcatheter assistance, a knife can enter an eye through a transparent corneal incision. An inner wall of a Schlemm's canal and a trabecular meshwork were cut all around with the microcatheter by cutting a part of the trabecular meshwork, such that a resistance of an inner drainage pathway of aqueous humor was reduced and intraocular pressure was reduced. The outflow of the aqueous humor was independent of a filtering bleb to avoid the complication of the filtering bleb.

### 2.2 External Drainage Surgery:

The trabeculectomy was combined with the Schlemm outer wall incision, namely, a 1*3 mm trabecular tissue was incised, aqueous humor was drained to the subconjunctival space, and meanwhile, a Schlemm's canal outer wall about 1 mm was incised under a deep scleral flap. Therefore, the aqueous humor can enter the Schlemm's canal, a part of the aqueous humor entered an internal drainage, but the effect was limited, and the external drainage effect of trabecular excision was mainly used.

### 2.3 Penetrating Schlemm's Canalotomy:

The penetrating Schlemm's canalotomy was a multi-pathway drainage surgical method. On the basis of ab externo 360° Schlemm's canalotomy, trabecula of about 1*1.5 mm was incised, such that the aqueous humor was drained along both an aqueous humor outflow pathway and a subconjunctival drainage pathway of an internal pathway. The success rate of the surgical method was higher than that of the 360° trabeculotomy with glaucoma gonioscope and microcatheter assistance, significantly reducing the probability of intraocular pressure rebound and a lower degree of intraocular pressure elevation.

e.g.: Table 1 Comparison of surgical effects

| | Internal | External | Surgery of |
|---|---|---|---|
| | drainage | drainage | the present |
| | operation | operation | disclosure |
| Total cases | 11 | 13 | 16 |
| Successful surgeries | 8 | 11 | 14 |
| Postoperative | 27.2% | 15.4% | |
| intraocular pressure | | | 12.5% |
| rebound rate | 72.7% | 84.6% | 87.5% |
| Surgical success rate | | | |
| Filtering bleb | 0 | 30.8% | 18.8% |
| complication rate | | | |

It can be seen from Table 1 that in the surgical treatment of the patients with childhood glaucoma in our hospital, the effective rate of the 360° trabeculotomy with gonioscope and microcatheter assistance was 72.7%, the postoperative intraocular pressure rebound rate was 27.2%, and the duration was 3-14 days. The treatments involved intraocular pressure-reducing medications and anterior chamber puncture mainly used, wherein 2 patients were subjected to an anterior chamber irrigation. The effective rate of the internal drainage operation reported by the literature was 68-90% and the postoperative intraocular pressure rebound rate was about 37%. The internal drainage surgery was mainly used in the patients with glaucoma in early and middle stages. This procedure requires a clear cornea, and for children with poor corneal clarity, an anterior chamber surgery under the gonioscopy was difficult to perform, thus the ab externo Schlemm's canalotomy can be performed. Its effect was similar to that of the internal surgery. The success rate of the external drainage operation was 84.6%, but the incidence rate of a complication of a filtering bleb reached 30.8%, which had a certain influence on the quality of life of child patients. The penetrating Schlemm's canalotomy, as provided in the present disclosure, had a success rate of 87.5% and a rate of intraocular pressure rebound of 6.3% lasting 1-2 days. The intraocular pressure can be reduced by massaging eyeballs, removing adjustable sutures, etc. The treatment was relatively simple and had lower probability of the complication of the filtering bleb than that of the external drainage operation. Therefore, for patients with advanced childhood glaucoma, the risk of further visual function damage or even blindness caused by intraocular pressure rebound was obviously reduced. Thus, for childhood glaucoma, especially in patients with serious visual function impairment, the surgical method of the present disclosure is an ideal surgical option.

What is claimed is:

1. A surgical method for Schlemm's canalotomy, comprising the following steps:
S1, performing anesthesia: performing anesthesia by retrobulbar anaesthesia;
S2, fixing an eyeball: pulling a superior rectus by using a 5-0 silk suture, and for a patient with a larger diameter of cornea, suspending a corneal limbus by using a 6-0 absorbable suture;
S3, creating a conjunctival flap: forming a conjunctival flap with a fornix as a base, cutting a full layer of a bulbar conjunctiva and a subconjunctival tissue at the corneal limbus along the corneal limbus, performing a blunt dissection by closely attaching the surface of a sclera under a fascia Tenon capsule to expose a surgical field, and performing a blunt dissection from an incision to a conjunctival fornix;
S4, performing hemostasis in a surgical area: performing hemostasis on the sclera surface of the intended scleral flap by using an underwater electrocoagulation method to avoid excessive electrocoagulation;
S5, creating a superficial scleral flap: creating a superficial scleral flap based at the corneal limbus, measuring 4 mm×3 mm and approximately half the thickness of the sclera; and dissecting forward to the transparent corneal limbus, performing hemostasis in the scleral flap area again and placing mitomycin under the conjunctiva and the scleral flap;
S6, puncturing an anterior chamber: making an auxiliary incision in the transparent cornea within the corneal limbus in the upper nasal or upper temporal area by using a puncture knife to keep an intraocular pressure at slightly lower than a normal level, and injecting a miotic and a small amount of a viscoelastic agent into the anterior chamber to reduce the risk and degree of outward bulging of the inner wall of Schlemm's canal;
S7, making a deep scleral flap: making a deep scleral flap in the central area of a scleral bed beneath the superficial scleral flap and gradually dissecting forward; when ring-like fibers appear in the scleral bed, continuing the dissection until the outer wall of Schlemm's canal is incised, exposing the gray-brown inner wall of Schlemm's canal;
S8, penetrating the Schlemm's canal: connecting a microcatheter with an illumination indicator to a viscoelastic injector and rotating the injector until the viscoelastic agents emerge from the tip of the microcatheter; dimming the microscope light, gently clamping the microcatheter by a pair of microscopic forceps, inserting the microcatheter into an opening of the Schlemm's canal, slowly advancing the microcatheter along the corneal limbus, and monitoring the position of indicator lamp at the tip of the microcatheter to ensure it remains within the lumen of Schlemm's canal until the microcatheter exits from the opposite end;
S9. 360° cutting the Schlemm's canal: pulling both ends of an optical fiber and 360° cutting the inner wall of the Schlemm's canal, wherein anterior chamber bleeding can be generally seen;
S10, suturing the deep scleral flap: suturing the deep scleral flap with a 10-0 nylon suture to prevent excessive filtration during and after surgery, which leads to complications of low intraocular pressure and a shallow anterior chamber;
S11, excising a trabecular tissue: puncturing into the anterior chamber from the anterior edge of the Schlemm's canal under the superficial scleral flap by using a puncture knife and then completely excising corneal and scleral tissues in parallel to the corneal limbus;
S12, excising a peripheral iris: making a wide base peripheral iris excision parallel to the corneal limbus, wherein an excision area is larger than a trabecular incision;
S13, suturing the scleral flap and the conjunctival flap: tightly suturing the anterior edge of the scleral flap with a 10-0 nylon suture, injecting a balanced salt solution or sterile normal saline into the anterior chamber from the corneal auxiliary puncture site to reform the anterior chamber and checking for fluid leakage condition at the scleral flap by using a sponge stick or a cotton swab; and suturing the conjunctival incision by using a 10-0 nylon suture in a horizontal mattress manner; and
S14, completing the surgery: removing the superior rectus traction suture or the corneoscleral limbus suspension suture, removing an eyelid speculum, applying an eye antibiotic and glucocorticoid-containing ointment in a conjunctival sac, covering the operated eye with a dressing, and completing the procedure.

2. The surgical method according to claim 1, wherein in step S1, general anesthesia is performed for a patient with claustrophobia or excessive anxiety.

3. The surgical method according to claim 1, wherein in step S3, the full layer of the bulbar conjunctiva and the subconjunctival tissue are cut using conjunctival scissors along the limbal, 1 mm outside the corneal limbus; the length is to be between 6-9 mm, preferably 8 mm; and during blunt separation on the scleral surface, the surgical field of 5 mm×4 mm is exposed.

4. The surgical method according to claim 1, wherein in step S5, the superficial scleral flap is 4 mm×3 mm in size and half the thickness of the sclera.

5. The surgical method according to claim 1, wherein in step S5, dissecting forward is performed to approximately 0.5-2 mm within the transparent corneal limbus, preferably 1 mm; and mitomycin is placed under the conjunctiva and the scleral flap for 1-4 minutes, preferably 2 minutes.

6. The surgical method according to claim 1, wherein in step S6, the 15° puncture knife is used as an auxiliary knife to create an auxiliary incision in the transparent cornea 0.5 mm inside the corneal limbus.

7. The surgical method according to claim 1, wherein in step S7, the deep scleral flap with the size of 1.5 mm×2 mm is made in the central area of the scleral bed below the superficial scleral flap and the depth is such that only a few fibers remain in the scleral bed and the gray-blue choroid membrane can be seen.

8. The surgical method according to claim 1, wherein in step S8, when the Schlemm's canal is penetrated, if resistance is encountered, the microcatheter can be slightly retracted and a small amount of the viscoelastic agent is injected to expand the Schlemm's canal and then the microcatheter is advanced continuously; if the microcatheter get stuck, the microcatheter can be retracted into the Schlemm's canal and pass through again, and if the microcatheter is still stuck, the microcatheter can be withdrawn and reinserted from the opposite end of Schlemm's canal; and if full penetration still cannot be performed, a 360° Schlemm's canalotomy of an internal approach can be considered.

9. The surgical method according to claim 1, wherein in step 11, the 15° puncture knife punctures into the anterior chamber from the anterior edge of the Schlemm's canal under the superficial scleral flap and then 1.0 mm×0.5 mm of the corneal and scleral tissues are completely excised in parallel to the corneal limbus.

10. The surgical method according to claim 1, wherein in step S13, the anterior edge of the scleral flap is tightly sutured with 2 stitches, 2 or more adjustable sutures are used for suturing, the balanced salt solution or sterile normal saline are injected into the anterior chamber from the auxiliary puncture site of the cornea to reform the anterior chamber and the fluid leakage condition is checked at the scleral flap by using a sponge stick or cotton swab using the stable anterior chamber and no obvious leakage existing at the scleral flap part as the criterion; and the conjunctival incision is sutured by using the 10-0 nylon suture in a horizontal mattress manner.
